(19) United States
(12) Patent Application Publication
(10) Pub. No.: US 2011/0178016 A1
(43) Pub. Date: Jul. 21, 2011

(54) PULSE PARATHYROID HORMONE FOR TREATMENT OF THE HEMATOPOIETIC SYNDROME, STROMAL CELL LOSS, AND VASCULAR INJURY RESULTING FROM ACUTE EXPOSURE TO LETHAL RADIATION

(75) Inventors: Amelia Bartholomew, Chicago, IL (US); Eduardo Reina, Chicago, IL (US); Alex Lyubimov, Chicago, IL (US); Dan Cramer, Chicago, IL (US)

(73) Assignee: The Board of Trustees of the University of Illinois, Urbana (US)

(21) Appl. No.: 13/121,981

(22) PCT Filed: Sep. 30, 2009
(86) PCT No.: PCT/US09/59085

§ 371 Date: Mar. 31, 2011

Related U.S. Application Data
(60) Provisional application No. 61/101,900, filed on Oct. 1, 2008.
(60) Provisional application No. 61/174,932, filed on May 1, 2009.
(60) Provisional application No. 61/232,327, filed on Aug. 7, 2009.
(60) Provisional application No. 61/242,013, filed on Sep. 14, 2009.

(51) Int. Cl. A61K 38/29; C07K 14/635; A61P 43/00; A61P 7/00; A61P 37/04

(52) U.S. Cl. 514/11.8; 530/399

(57) ABSTRACT

Currently there is no treatment for hematopoietic syndrome following radiation exposure. Exposed persons are presently treated with blood transfusions, growth factors such as G-CSF to promote neutrophil recovery. Present methods are targeted towards the bone marrow microenvironment, aiding in repair and regeneration with a decrease in the severity or possibly, complete avoidance of morbidity and mortality associated with the hematopoietic syndrome. Methods are useful for treatment of mass casualties following a radiation disaster.

### BACKGROUND

Relatively high doses of parathyroid hormone (PTH) are administered to mammals for a short period close in time to exposure of the mammals to acute lethal levels of radiation or to chemotherapy, in order to prevent or treat resulting hematopoietic syndrome, bone marrow stromal cell loss, and vascular injury, thereby increasing survival time.

In 2004, the Secretary of the Department of Homeland Security (DHS) of the United States highlighted the potential of the radiological and nuclear threat. At present medical countermeasures against a radiological threat are limited. Recovery from the lethal effects of irradiation across the hematopoietic system requires critical levels of cell and tissue regeneration. Early survival of exposed mammals will be dependent on the regeneration and recovery of pluripotential, multipotential and committed stem and progenitor cells for short-term and sustained reconstitution of the hematopoietic system. The extent of recovery will dictate the numbers of neutrophils and platelets available to prevent the morbidity and mortality associated with the consequent hemorrhage and sepsis. The bone marrow stroma plays a critical role in support of both hematopoiesis as well as stromal cell availability for repair of tissues, including the vasculature. Measures aimed at regeneration of the bone marrow microenvironment may improve support for hematopoiesis as well as mitigate damage to vasculature following damaging radiation or chemical exposure.

For example, in response to acute radiation exposure of 0.7-10.0 Gy, an ensuing hematopoietic syndrome can include anemia, thrombocytopenia, and infectious complications associated with neutropenia. Treatment, consisting of intravenous fluids, blood products and aggressive anti-microbials can significantly improve mortality, however the deployment of blood products for platelet support in a mass casualty situation, may not be feasible. Following a nuclear attack, it is estimated that 24% of the blood supply would be destroyed, with diminished ability for replenishment, due to diminished available donors, disrupted transportation, and reduced availability of medical personnel. Greater than 70% of radiation victims are projected to sustain trauma and/or burn injuries, also requiring blood product support, further draining limited resources. Another problem is that the efficacy of platelet transfusions may be diminished due to the presence of fever, bleeding, sepsis, neutropenia, and the potential need to transfuse ABO incompatible units, all of which reduce platelet lifespan.

Parathyroid hormone (PTH) has been demonstrated to lead to hyperproliferation of spindle-shaped N-cadherin⁺CD45⁻ osteoblasts (SNO) that influence long-term repopulating hematopoietic stem cells (HSCs) through secretion of bone morphogenetic protein and Notch ligand jagged 1 signaling. Notch is present on HSCs, and signaling has been shown to increase stem cell numbers without expanding mature cells. Increased HSC production may not be the only possible explanation by which PTH influences megakaryopoiesis. Osteoblasts, activated by PTH or the locally produced, PTH-related protein (PTHrP), through the PTH/PTHrP receptor (PPR), may produce hepatocyte growth factor (HGF), which has a direct involvement in megakaryocyte production. HGF enhances production of thrombopoietin and SDF-1, which stimulates the ex vivo differentiation of CD41+ megakaryocytes and platelets. HGF can also affect the production of T and B cell lineages. PTH treatment led to an increase in T and B lineages as well as HSC, suggesting osteoblast-production of HGF may also play a role. PTH could induce osteoblast control of platelet production through increased production of HSC by niche expansion, or through increased megakaryocyte differentiation, possibly via HGF.

### SUMMARY

Methods and compositions described herein are useful for treatment of mass human casualties following a radiation disaster and for prevention and treatment of hematopoietic syndrome, stromal cell loss and vascular injury resulting from radiation exposure or chemotherapy.

Parathyroid hormone (PTH) has a beneficial effect on reconstitution of hematopoiesis following bone marrow ablation with chemotherapy. Surprisingly, this beneficial effect was similarly observed following tissue injury to the bone marrow microenvironment that occurs as a consequence of radiation exposure. PTH provides a survival advantage in response to a lethal irradiation dose expected to result in 50% death of exposed mice. Daily or twice doses of PTH resulted in 100% survival. PTH also protects against infection in the survivors.

Parathyroid hormone administered at high doses in pulses, is useful to treat mammals exposed to acute, lethal doses of radiation such as expected in a “dirty bomb,” e.g. 0.7-10.0 Gy. The sooner after exposure PTH is administered, the better the prognosis, and the greater chance for survival.

A parathyroid hormone treatment is correlated with production of hematopoietic stem cells, and stimulates production of CD34⁺ cells.

A fragment of PTH corresponds e.g. to an N-terminal region of human parathyroid hormone that includes about 1-34 amino acids. The fragment may be recombinant or synthetic. In an embodiment, the fragment is teriparatide.

The parathyroid hormone is administered intranasally, subcutaneously or orally, in a dose range of about 1.4 μg/kg/day to 250 μg/kg/day.

PTH may be administered for a brief time period ranging from 20-50 days, preferably for about 30 days following an exposure to acute radiation.

Materials and methods are suitable for radiation doses anticipated to result in, for example, 10-90% mortality in 60 days, following exposure.

A method of increasing the survival rate in a mammal exposed to acute radiation, includes:

(a) obtaining a pharmaceutical composition comprising a therapeutically effective amount of parathyroid hormone (PTH) or a biologically active fragment thereof; and

(b) administering the composition to the mammal to increase the survival rate of the mammal.

A method of increasing platelet production in a mammal following a radiation exposure, includes:

(a) obtaining a pharmaceutical composition including a therapeutically effective amount of parathyroid hormone (PTH) or a biologically active fragment thereof; and

(b) administering the composition in pulses to reduce the degree of thrombocytopenia in the mammal. Pulses are defined herein as high doses of the composition administered in a short duration, with serum PTH levels returning to baseline (prior to PTH administration levels) within 3-4 hours of administration. A therapeutically effective dose is several orders of magnitude higher than doses of PTH used for other applications e.g. treatment of osteoporosis.

A method of mitigating acute radiation syndrome in a mammal exposed to acute radiation, includes:

(a) obtaining a pharmaceutical composition that includes a therapeutically effective amount of parathyroid hormone (PTH) or a biologically active fragment thereof; and

(b) administering the composition to reduce radiation induced acute vascular injury in the mammal, thereby reducing end organ damage, such as reducing and/or eliminating intestinal barrier dysfunction.

A method to improve vascular repair and reduce end organ damage by alleviating acute vascular injury to a mammal exposed to radiation includes:

(a) obtaining a pharmaceutical composition comprising a therapeutically effective amount of parathyroid hormone (PTH) or a biologically active fragment thereof; and

(b) administering the composition to the mammal.

A method to improve the ability of bone marrow stroma to support immune function post radiation of a mammal by enhanced T cell and macrophage reconstitution, particularly the cells with a CD4+ phenotype includes:

(a) obtaining a pharmaceutical composition comprising a therapeutically effective amount of parathyroid hormone (PTH) or a biologically active fragment thereof; and

(b) administering the composition to the mammal.

A method to reduce risk of infection in survivors of lethal doses of radiation, includes:

(a) obtaining a pharmaceutical composition comprising a therapeutically effective amount of parathyroid hormone (PTH) or a biologically active fragment thereof; and

(b) administering the composition to the survivors.

A pharmaceutical composition described herein includes a therapeutically effective amount of parathyroid hormone (PTH) or a biologically active fragment thereof, wherein the PTH or the fragment thereof is capable of treating hematopoietic syndrome by e.g. mitigating thrombocytopenia, enhancing hematopoiesis, mitigating acute vascular injury and as a consequence of one or the combination of the above increasing survival rate in response to acute radiation injury. The PTH or the fragment thereof may be delivered by means of an aerosol formulation for a nasal spray application, injected subcutaneously, or ingested, or other equivalent methods to achieve transient high PTH levels in the serum.

An embodiment of a therapeutically effective amount of PTH is in a range of about 1.4 μg/kg/DA4 to about 250 μg/kg/day as single or divided doses. Other doses depend on the activity of the composition and may vary to produce effects on hematopoietic cells, in particular, stem cells.

Strategies aimed at increasing endogenous platelet lifespan or increasing production are beneficial. Parathyroid hormone (PTH) is a non-toxic agent capable of improving platelet production, and CD34+ cell production in primates. PTH rapidly metabolizes so effects on target cells disappear after 4-6 hours. If insufficient PTH is administered to cells, they are unable to initiate the repair mechanism.

Monkeys and mice are used herein as mammalian models. In monkeys, the first 30 days are important, a finite response. The first 1-14 days are when the most potent effects of PTH occur. An aspect of the disclosure is to establish the range of efficacy of PTH following a range of lethal acute radiation exposures.

Parathyroid hormone (PTH) stimulation has been demonstrated to lead to increased numbers of hematopoietic stem cells (HSCs), presumably through a targeted effect on the hematopoietic niche, leading to expansion of the number of niches. An increase in the number of hematopoietic stem cells could mitigate the myelosuppressive interval following irradiation, thereby leading to diminished duration of thrombocytopenia, higher levels of platelet counts, with a corresponding decreased utilization of blood transfusions during the 60 day period following exposure to radiation. A decrease in the duration of thrombocytopenia may also reduce acute mortality due to a decrease in hemorrhagic complications.

The PTH containing compositions described herein may also be used with one or more additional agents that may stimulate or assist in the stimulation of hematopoietic system by PTH following acute radiation exposure such as IL-3, or HGF.

Toxicity studies in monkeys indicated that PTH was tolerated at doses comparable to doses to be used to ameliorate radiation damage. Tolerance was observed in both irradiated and non-irradiated monkeys.

### BRIEF DESCRIPTION OF THE DRAWINGS

FIG. 1 is a graphical representation of platelet counts in peripheral blood of macaques treated with PTH compared to untreated controls; results demonstrate a statistically significant increase in platelet counts first observed 7 days after PTH therapy and persisting after radiation exposure.

FIG. 2 shows graphically, effects of PTH on T cells; Following a bone marrow transplant with a complete MHC mismatched donor, engrafting donor T cells identified by the expression of the class I marker BW6, was quantitated over time (top panels) revealing significant increases in both CD4 and CD8 subsets; host T cells (lower panels) had no significant increase.

FIG. 3 shows graphically, CD34+ counts in PTH treated animals and controls; the top graph shows the number of engrafting donor CD34+ cells in PTH treated and untreated animals; the lower graph shows increases of host CD34+ cells above baseline pre-treatment levels following PTH therapy.

FIG. 4 are photomicrographs of bone biopsies of monkeys 7150 and 7154 taken before (pre) and 5 weeks after (post) PTH treatment; pre-PTH treatment shows marked adiposity; post-treatment there is increased cortical bone, increased osteoblasts, and loss of adiposity.

FIG. 5 shows increased survival of mice exposed to lethal radiation (772 cGy) and 24 hours later underwent a 28 day course of PTH 80 mcq/kg/days.

FIG. 6 shows results showing percent survival of mice irradiated with a lethal dose (772 cGy); comparison of mice treated with 200 μg/day, 200 μg twice daily (BID) vs no PTH treatment.

FIG. 7 shows 49 day survival of mice treated with various doses of PTH at 400 μg/28 day, 1000/400/7 days, 400 at 7 days.

FIG. 8 shows CFU in mice euthanized on day 7.

FIG. 9A shows CFU-GM following radiation exposure of mice and PTH treatment at day 3 and day 7, compared to no PTH treatment.

FIG. 9B shows CFU-M following radiation exposure of mice and PTH treatment at day 3 and day 7, compared to no PTH treatment.

FIG. 10A shows BFU following radiation exposure of mice and PTH treatment at days 3 and 7.

FIG. 10B shows CFU-GEMM at 3 and 7 days following radiation exposure of mice and daily PTH treatment, compared with no PTH treatment.

FIG. 11 shows bone marrow hematopoietic stem cell content following radiation exposure in mice followed by treatment with daily PTH or saline.

FIG. 12 shows PTH levels in serum of mice treated with 400 mcg/kg PTH assayed after the final PTH dose on day 28.

FIG. 13A shows monkey (CYNO) CFU-GM by PTH dose, pre and post 24 hours treatment.

FIG. 13B shows monkey (CYNO) CFU-M by PTH dose, pre and post 24 hours treatment.

FIG. 14A shows monkey (CYNO) BFU by PTH dose.

FIG. 14B shows monkey (CYNO) CFU-GEMM by PTH dose.

FIG. 15 shows CD34+ hematopoietic stem cell content by PTH dose in non-irradiated CYNOS (monkeys).

FIG. 16 (A), (B), (C), and (D) shows subjects (monkeys) 7801 (100 mcg/kg PTH) and 7872 (50 mcg/kg PTH) underwent MRI examination of the kidneys upon completion of a 21 day course of PTH therapy. Notably, no cystic degeneration or tumors were found in the kidneys. No change was detected in BUN or creatinine to indicate compromise in renal function.

FIG. 17 shows a comparison of serum calcium levels of three non-irradiated cynomolgus monkeys treated with escalating doses of PTH for 21 days: 7872 (50 ug/kg), 7801 (100 ug/kg), 7876 (250 ug/kg). While slightly elevated, the animals did not experience toxicity during the first 20 days of dosing. Animal 7876 showed some somnolent behavior on the 21^{st} day of dosing which also corresponded to a calcium level of 17 mg/dl. These data indicated 250 ug/kg up to 19 days is well tolerated.

FIG. 18 shows serum calcium levels of 7875 and 7801 showed normal calcium levels except one timepoint, day 19 for 7801, with subsequent time points falling within normal range.

FIG. 19 shows a serum calcium levels in 7801 following PTH 100 mcg/kg/d and following 686 cGy exposure and PTH 100 mcg/kg/d.

FIG. 20A shows T wave height at ST-intervals at the highest dose, 250 ug/kg.

FIG. 20B shows T wave height at the highest dose, 250 ug/kg, administered for 21 days in an irradiated animal shows some decrease in both over time. No life-threatening arrhythmias were detected indicating this high dose of short duration is a clinically feasible dose.

FIG. 21 shows serum levels of PTH from two non-irradiated monkeys, 7801 (100 mcg/kg/d) and 7872 (50 mcg/kg/d) performed for PK analysis on days 1 and 18 during PTH dosing. PTH becomes undetectable by 4-6 hours after dosing, despite extremely high doses of PTH.

### DETAILED DESCRIPTION

Pulse PTH therapy increased survival rates after lethal radiation exposure of mammals. Increased platelet counts, a feature important in mitigating regimen related thrombocytopenia, increased donor and autologous CD34+ cell numbers, and improved donor lymphoid chimerism, all of which are desirable goals in host tolerizing regimens, contributed to survival.

PTH Composition

The term “parathyroid hormone” or “PTH” as used herein includes naturally occurring full-length (1-84 amino acids), partial fragments, truncated versions (e.g., 1-34 N-terminal amino acids), synthetic, recombinant, derivatives, analogs of human parathyroid hormone that are biologically active (comparable to full-length or truncated 1-34 fragment) to induce hematopoietic and stromal cell proliferation following an acute radiation exposure. The term “parathyroid hormone” also encompasses variants and functional analogs of PTH. Thus, pharmaceutical formulations that include PTH variants and functional analogues with one or more modifications such as substitutions, deletions, insertions, inversions or cyclisations, yet having substantially the biological activities of parathyroid hormone, are included. Stability-enhanced variants of PTH known in the art are also included (e.g., U.S. 5382658; WO93/020203). Variants of PTH may incorporate amino acid substitutions that improve PTH stability and half-life, such as the replacement of methionine residues at positions 8 and/or 18, and replacement of asparagine at position 16.

Native human parathyroid hormone (PTH) is an 84 amino acid polypeptide that is normally secreted from the parathyroid glands. Human PTH including its biologically active fragments thereof may be obtained through peptide synthesis or recombinantly from genetically engineered yeast, bacterial or mammalian cell hosts. Human PTH or fragments thereof including synthetic and recombinant versions are commercially available from a number of commercial vendors including for example FORTEO® [teriparatide (rDNA origin)] containing recombinant human parathyroid hormone (1-34), [rhPTH(1-34)] by Eli Lilly and Co., (Indianapolis, Ind.) and a synthetic version by Merck Sharp and Dohme, (West Point, Pa.). Fragments of PTH incorporate at least the amino acid residues of PTH necessary for a biological activity similar to that of intact PTH. Examples of such fragments include for example PTH(1-31), PTH (1-32), PTH (1-32), PTH (1-33), PTH(1-34), PTH(1-36), PTH(1-37), PTH(1-38), PTH(1-41), PTH(28-48), PTH(1-25) variants and PTH(25-39).

Suitable PTH analogs for use in accordance with the present invention include those described in U.S. Pat. Nos. 5,723,577, 5,861,284, 5,589,452, 5,849,695, 5,695,955, 6,362,163, 6,147,186, 6,472,505 and 6,583,114; US 20080176787, US 20060270603, the disclosures of all of which are hereby incorporated by reference in their entirety. Cyclized PTH analogues are also described in U.S. Pat. Nos. 5,556,940; 5,955,425; 6,110,892; 6,316,410; and 6,541,450 the disclosures of all of which are hereby incorporated by reference in their entirety.

Teriparatide (1-34 PTH) formulations (e.g., a recombinant version sold by Eli Lilly as FORTEO®) may be described in one or more U.S. patents including U.S. Pat. No. 6,590,081, U.S. Pat. No. 6,770,623, and U.S. Pat. No. 7,144,861, the disclosures of all of which are hereby incorporated by reference in their entirety.

Administration of PTH

PTH formulations including pharmaceutical compositions are administered through any acceptable mode including for example, oral, sub-lingual, nasal, subcutaneous, transdermal, intravenous, intramuscular, intraperitoneal, intracavity, pulmonary, and inhalation. Suitable pharmaceutical formulations include liquids, solids, tablets, pills, capsules, injections, aerosols, dry powder inhalation, gels, pastes, and the like. When administered, pharmaceutical preparations of the invention are applied in pharmaceutically acceptable amounts and in pharmaceutically acceptable compositions. Such preparations may routinely contain salts, buffering agents, preservatives, compatible carriers, and optionally other therapeutic ingredients. When used in medicine the salts should be pharmaceutically acceptable, but non-pharmaceutically acceptable salts may conveniently be used to prepare pharmaceutically acceptable salts thereof and are not excluded from the scope of the invention.

Nasal administration of PTH and analogs thereof are described for example in U.S. Pat. No. 7,244,709, U.S. Publication Nos. 20080119408, 20080051332, 20070244049, and 20060127320, assigned to Nastech Pharmaceuticals Inc. (Bothell, Wash.), the disclosures of which are incorporated by reference in their entirety.

Doses

Suitable dosages of PTH for rejuvenating the hematopoietic system following an acute radiation exposure include for example about 1.4 μg/kg/day to about 250 μg/kg/day of PTH (e.g., teriparatide) for a period of about 21-50 days. In particular specific doses may include 1.5, 2.0, 2.5, 3.0, 3.5, 4.0, 4.5, 5.0, 5.5, 6.0, 6.5, 7.0, 7.5, 8, 8.5, 9, 9.5, 10, 10.5, 11, 11.5, 12, 12.5, 13, 13.5, 14, 14.5, 16, 16.5, 17, 17.5, 18, 18.5, 19, 19.5, 20, 20.5, 21, 21.5, 22, 22.5, 23, 23.5, 24, 24.5, 5 to 250 μg/kg/day of PTH (e.g., teriparatide) for a subject exposed to acute radiation. If a synthetic analog or a variant of PTH that is more active than teriparatide (e.g., has a longer residence time in the body or has higher affinity for PTH receptor), appropriate reductions in dosage of the PTH analogs or variants are adopted by a skilled artisan such that the activity is equivalent to the activity exhibited by teriparatide dosages disclosed herein. Similarly, if an analog or a variant has lower relative activity compared to teriparatide, appropriate increases in the dosages are adopted by a skilled artisan to account for equivalent biological effects. The exact dosages used may be adjusted appropriately to achieve desired drug levels, local or systemic, depending upon the particular properties of the PTH molecule administered, including its molecular weight and stability, and the mode of administration. For example, an intravenous administration may have a lower dose per day compared to the oral doses. Similarly, nasal administrations may involve a 10-20 fold higher dose (e.g., teriparatide) as long as such administrations are not toxic to the individuals. Higher doses than those described herein including for example, 26 or 27 or 28 μg/kg/day of PTH (e.g., teriparatide) may be administered as long the dose is not toxic to humans over a 20, 30, 40, or 50 day period.

Based on the dosage information provided herein, multi-use format of pharmaceutical products that deliver for example, about 50 μg to 1875 μg in a single dose or in divided doses for about 21-50 days, are suitable.

Pulsed Doses

A single dose of PTH can diminish mortality following exposure to lethal irradiation. As early as four hours following PTH injection, mitotic bone marrow cells were observed within the bone marrow, and were identified as cycling shot-term reconstituting spleen colony forming cells (CFU-S). One mechanism by which PTH enhances survival following irradiation, may be the initiation of DNA replication of cycling CFU-S cells that have exited the primitive quiescent, G₀, niche-bound long-term repopulating (LTR)-HSC cell state and these cells may increase megakaryocyte production. Notably, at the low dose tested no effect was observed when PTH was given after radiation, leading to abandonment of this treatment. Initiation of this effect may be due to the presence of PTH receptors on hematopoietic cells themselves because expression of the PTH receptor has been detected on these cells. PTH treatment may stimulate HSC lineages directly, thus providing direct control over platelet production.

The term “pulse” or “burst” as used herein means administering a high-dose of PTH followed by a rest-period and then a subsequent dose or doses of PTH. The therapeutic potential of PTH may be optimized through brief exposure to PTH via pulsed dosing instead of continuous infusion. In infusions of PTH lasting greater than 1 hour, detrimental effects were observed on osteoblasts, with associated hypercalcemia and weight loss in vivo. Patients treated with constant infusion of PTH developed diminished osteoblast activity. Last, in patients with persistent hyperparathyroid levels, no beneficial effect is observed on hematopoiesis. In summary, the efficacy of PTH as an intervention for acute radiation syndrome is critically dependent on pulse therapy in which the PTH levels return to baseline within 3-4 hours or earlier following administration.

For example, a dose of about 7.0 μg/kg/day could be administered following radiation exposure with a second dose is administered about 12-24 hours later. In other words, a sustained presence of PTH in the system of an individual exposed to acute radiation is not desired and therefore a resting period is introduced between two consecutive doses. Depending on the dose used, a longer resting period of e.g., 2 days may also be implemented. Similarly, if a lower dose is chosen (e.g., 1.5 μg/kg/day), then a shorter resting period may be implemented between consecutive doses. Depending on the dose, the severity of the radiation exposure, the mode of administration, and the nature of PTH formulation, the dose-rest-dose may vary from 12 hours to about 36 hours (i.e., twice daily to about once in 3 days). The treatment regimen may last for about 7, 10, 12, 14, 21, 25, 20, 36, 40, 50 and 60 days following acute radiation exposure. In addition, the dosing may be more frequent (i.e., daily) during the first 14 days of acute radiation exposure as compared to the later days (e.g., once in two days for days 30-50). Preferably, the dosage is daily and lasts up to 30-50 days following acute radiation exposure.

A murine model of radiation injury was used to determine the extent in which PTH mitigates acute radiation syndrome mortality, hematopoiesis, and thrombocytopenia. A range of PTH doses shown to be non-toxic in humans was tested to determine efficacy, however higher doses appear to have greater efficacy for hematopoietic effects. PTH, developed as therapy for osteoporosis, has been clinically tested by numerous studies over a wide range of clinically feasible PTH doses. Four clinically relevant dosing schedules of PTH tested, the two lower doses which have already been tested in Phase I and II clinical trials, a higher dose, equivalent to the dose used in nonhuman primate studies, and significantly higher doses not tested before in humans. The decision to use low PTH doses in clinical trials for osteoporosis is based on the need for long-term compliance (18-24 months) for treatment of this condition. Higher doses are associated with increased incidence of adverse events such as headache and nausea. However since a short 7-60 day course of the drug is proposed to increase survival after radiation, and PTH has potential to significantly impact morbidity and mortality in a mass casualty situation, testing these higher doses is an appropriate strategy.

The efficacy demonstrated for PTH-induced improvement on hematopoiesis, platelet production, and vascular stroma can potentially lead to most importantly, decreased mortality, and can significantly impact the number of platelet transfusions required in supportive care following a nuclear attack. A conservative set of statistics, generated by the resources available in the state of Michigan, estimated a pre-attack supply of platelets of 12,006 units would need to be increased 1,301 fold to address the post-attack need of 15,620,000 units. A 10% reduction in utilization (1,562,000 units) would be equivalent to 25% of the number of platelet units (4 million) transfused in the United States annually, and provide a significant savings in resources.

PTH is approved by the FDA, commercially available, and theoretically, could be immediately placed into a Strategic National Stockpile. Methods of delivery may include nasal spray and oral administration. Such methods of delivery are less invasive, leading to greater ease and less risk of administration, ideal for administration to large populations heterogeneous in age, ethnicity, and educational levels.

### EXAMPLES

### Example 1

### PTH Mitigates Myelosuppression Following Stem Cell Transplant and Improves Allogeneic Stem Cell Engraftment

A previously unrecognized role for PTH on mitigation of myelosuppression in macaque monkeys is described. Following chemo-ablative therapy and MHC mis-matched allogeneic stem cell transplant, animals were randomized to daily PTH treatment (5 mcg/kg/day subcutaneously) or placebo. Sequential peripheral blood analysis revealed daily PTH treatment significantly increased levels of host CD34+ stem cells as well as engrafting donor T, B and CD34+ cells. There was no significant increase in host or donor myeloid lineage CD14 or CD11b+ cells. Analysis of hemoglobin, neutrophils and platelets revealed increased levels of platelets, with statistical significance first observed by the 7th day of PTH treatment, with an increase in both CD34+ hematopoietic stem cells (HSC) and platelets. Following myeloablation, PTH may act to increase megakaryopoiesis through increased production of HSC.

### Example 2

### Parathyroid Hormone: Its Effects on Bone, Stem Cell Content, Progenitors, and Platelets

Parathyroid hormone (PTH) stimulation has been demonstrated to lead to increased numbers of HSCs, presumably through niche expansion. Bone marrow transplant studies by Calvi and colleagues characterized this process in the syngeneic mouse model of osteoblastic activation. To see if PTH could enhance allogeneic stem cell engraftment in nonhuman primates (monkeys), donors were MHC mismatched based on recipient proliferation against donor cells in the mixed lymphocyte culture and by disparity at the class I antigen, Bw6. This disparity permitted tracking of Bw6+ donor cells following transplantation into Bw6-recipients. Eight control recipients were conditioned with thymoglobulin, rituxan, fludarabine, and melphalan. Six animals received the conditioning regimen supplemented with parathyroid hormone beginning on the first day of the conditioning regimen and administered daily at a dose of 5 micrograms/kg subcutaneously. An allogeneic stem cell transplant consisting of 2−4×10⁶ CD34+cells/kg was administered on day 0. Outcome measures included PTH levels, CBCs, and donor engraftment via flow cytometry.

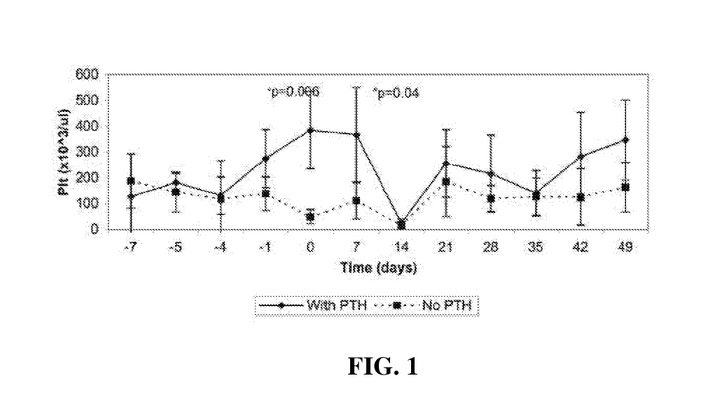

PTH levels peaked sharply 30 minutes after administration, with undetectable levels monitored by 4 hours. Serial CBCs revealed no significant difference when comparing hemoglobin between the two groups, suggesting that PTH did not affect red cell mass. There was a rapid rise in platelet counts in PTH treated animals, with significant increase observed 7 days after beginning PTH therapy (FIG. 1). It was not possible to determine whether PTH worked on HSC proliferation, HSC progenitor differentiation, or increased megakaryocyte differentiation. All three could be present alone or in combination.

No effect was observed on recipient granulocytes or natural killer cells. This observation was interesting given the current understanding that early HSC give rise to two distinct lineages, the common lymphoid progenitor (CLP) and the common myeloid progenitor (CMP); further differentiation of the myeloid progenitor gives rise to megakaryocyte/erythroid progenitors (MEP), which produce megakaryocytes. Lack of PTH effect on granulocytes could indicate PTH affected CMP differentiation into MEP, or PTH directed proliferating HSC to preferentially expand MEP, bypassing the CMP intermediary altogether.

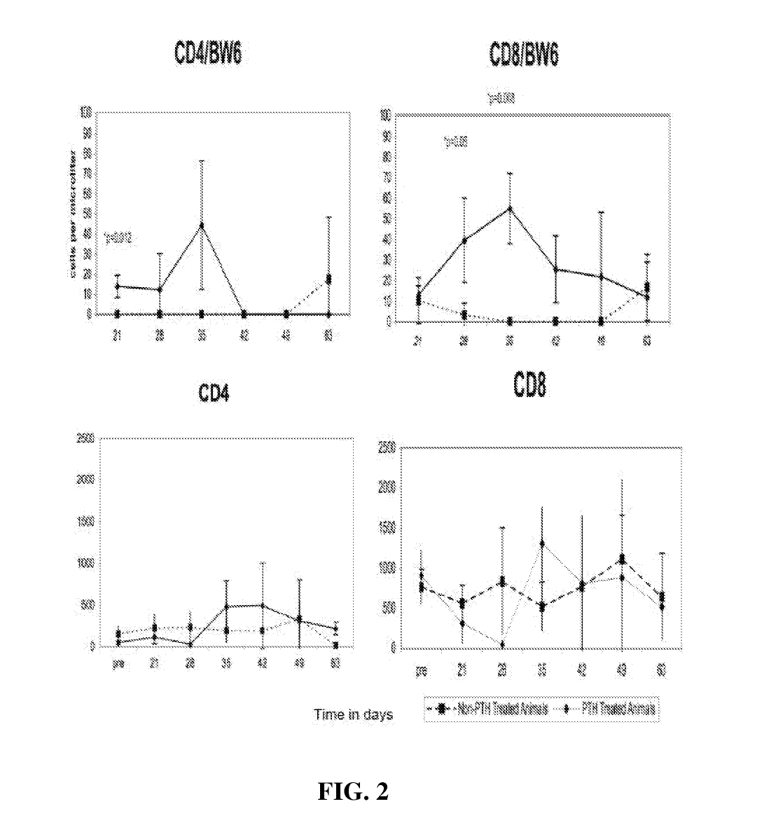

Both donor CD4 and CD8 subsets in PTH treated animals were increased over control animals with time (FIG. 2, upper panels). There was little or no effect on recipient T cells. The preferential effect of PTH on donor T cells could be due to several known effects PTH has on T cells: on CLP to spur differentiation into T cells, on T cell maturation by direct stimulation of thymic epithelial cells, or engagement of the PTH receptor directly on T cells, stimulating IL-2 production and its subsequent effects on facilitating T cell proliferation.

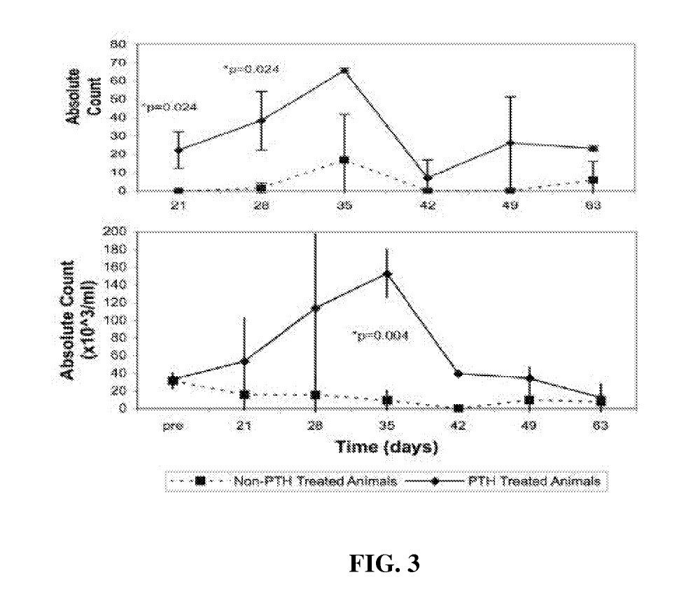

PTH induced significant increases in both donor and host CD34+ HSC. These differences were seen immediately, reached statistical significance by 4 weeks after treatment, and persisted after PTH had been discontinued on day 49 (FIG. 3). Early reports of PTH on bone marrow cells following irradiation showed increased numbers of cells with an increased mitotic activity, later identified as CFU-S, HSC progenitors of short-term populating ability. These may account for the early rise in CD34+ cells, since the CD34+ population contains both long and short-term repopulating cells.

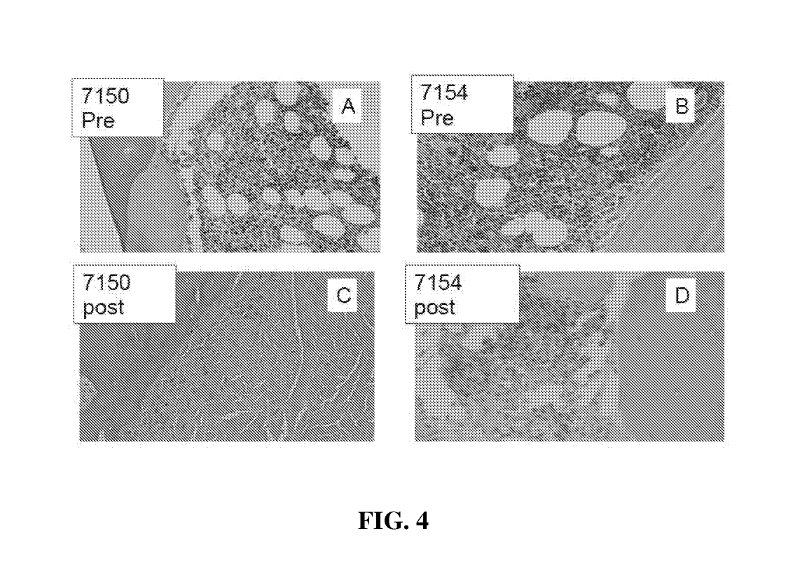

To characterize the effect of PTH on osteoblast formation, iliac crest bone biopsies were taken from animals treated with parathyroid hormone before and after therapy (FIG. 4). Prior to PTH treatment, adiposity within the bone marrow medullary component was observed. Following PTH treatment, adiposity was reduced, and cortical bone formation increased, with evidence of increased osteoblast proliferation.

Radiation reduces proliferative capacity of osteoblasts but does not appear to affect differentiation or linear growth. Irradiated mice developed a progressive decrease in the number of osteoblasts (38.1%), with osteoblasts only detectable on the surface of trabecular bone rather than existing along the long bone. Progressive fragmentation of trabecular structure corresponded with the reduction of SNO cells. Two weeks following 45 Gy limb irradiation, the PTH receptor was noted to be upregulated in hypertrophic and proliferative areas of the bone.

### Example 3

### Survival with PTH Treatment

In a study of 98 mice exposed to lethal irradiation, half received placebo (saline) and half received daily PTH 80 mcg/kg/day for 28 days. There was a survival advantage in the PTH treated group, 24/49 survived vs placebo, 16/49, (95% CI, 0.7707, 2.462).

### Example 4

### Effects of PTH Using a Murine Model of Radiation Exposure

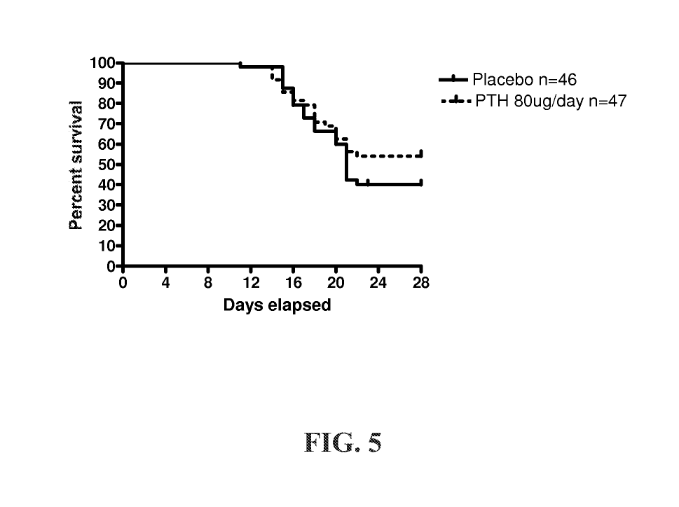

80 ug/day PTH therapy followed lethal irradiation (772 cGy) exposure. 49 mice were treated with daily PTH (80 mcg/kg/d sc×28 days) 24 hours following a lethal radiation exposure of 772 cGy and compared to 49 vehicle treated control animals. Outcome measures included platelet counts, survival, and other hematopoietic studies. When censoring for technical deaths, 46 animals were analyzed in the placebo group (daily saline injections) with 47 analyzed from the daily PTH 80 mcg/kg/day treatment. There was no significant difference in thrombocytopenia duration or nadir between the two groups. Thirty-day survival in the placebo group was 18/46 or 39%. Of the PTH treated animals 26/47 animals survived (55%, p=0.2). (FIG. 5) Although there was a trend for PTH to improve survival, this effect did not reach statistical significance using this dose of PTH.

### Example 5

### Experiments on 200 Micrograms/Kg/Day

In another set of dose finding experiments, the same radiation exposure was used (772 cGy). Three treatment groups were studied: (1) untreated (no placebo, n=8), (2) treated with 200 mcg/kg PTH daily beginning 24 hours after radiation exposure, n=12, (3) treated with 200 mcg/kg PTH twice daily beginning 24 hours after radiation exposure, n=12.

Comparison was made of animals treated with PTH, 200 ug/day, 200 ug twice daily (BID) vs those receiving no PTH treatment following lethal irradiation (772 cGy).

Three groups of B6 mice underwent irradiation.

Group 1 received no treatment, n=8.

Group 2 received PTH, 200 micrograms/kg/day, n=10, first begun 24 hours following radiation exposure to 772 cGy and continued for 28 days.

Group 3 received the same duration of PTH but instead was treated twice daily, 200 micrograms/kg/day BID for a daily total dose of 400 mcg/kg/day, n=12.

Thirty day survival was observed to be 50% for no treatment (4/8 alive), 67% (8/12) mice receiving a single dose PTH treatment, and 75% (9/12) in those receiving twice daily treatment.

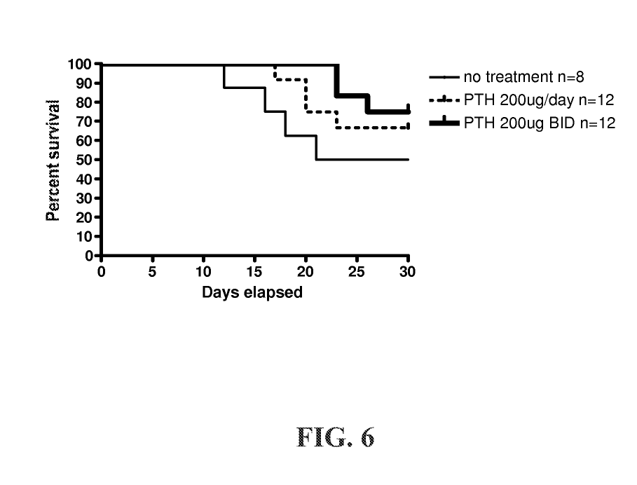

There was also a 25% reduction in mortality, changing the lethality of radiation exposure from an LD50/30 to an LD25/30. (FIG. 6)

### Example 6

### Experiments on 400 Mcg/Kg/Day

Four cohorts of B6 mice were irradiated to uniform, total body, midline tissue dose at a dose rate of 80+2.5 cGy/min to a dose of 772 cGy.

Group 1 received vehicle control (saline) treatment×28 days, beginning 24 hours after radiation, n=10.

Group 2 received PTH, 400 micrograms/kg/day, n=10, first begun 24 hours following radiation exposure to 772 cGy and continued for 28 days.

Group 3 received PTH, 1000 micrograms/kg/day, n=10, 24 hours following radiation, and then 400 mcg/kg/day for 7 days.

Group 4 received PTH, 400 micrograms/kg/day, n=10, 24 hours following radiation, and continued for 7 days.

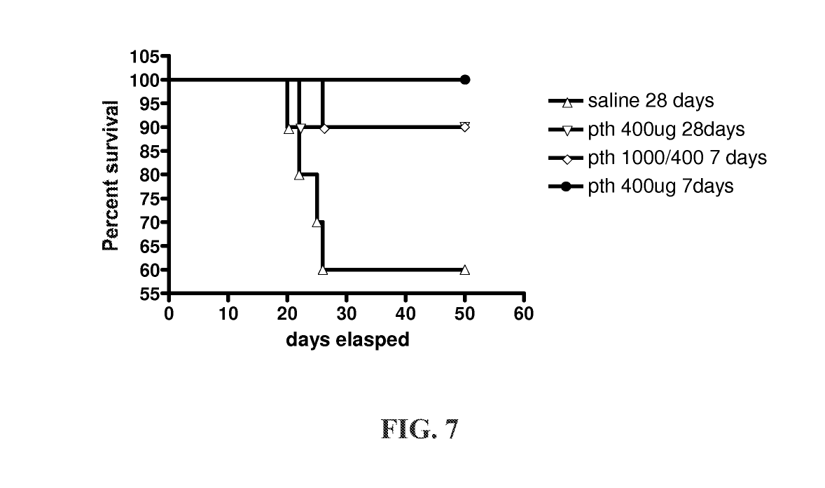

Shown in FIG. 7 is 49 day survival. Survival was observed to be 60% for vehicle control (6/10 alive), 90% (9/10) in 28 day PTH treatment and in high dose PTH followed by 7 day treatment (Groups 2 and 3, respectively, p=0.05 when compared to vehicle). PTH treatment, administered at the lower 400 mcg/kg day dose×7 days resulted in 100% (p=0.02 when compared to vehicle). No statistical difference was noted between groups 2, 3, 4 suggesting that a short course of lower dose PTH is equally effective to a 28 day course.

In a second experiment, a sub-group of animals were euthanized on day 7, n=3 per cohort. Bone marrows were flushed and CFUs were performed.

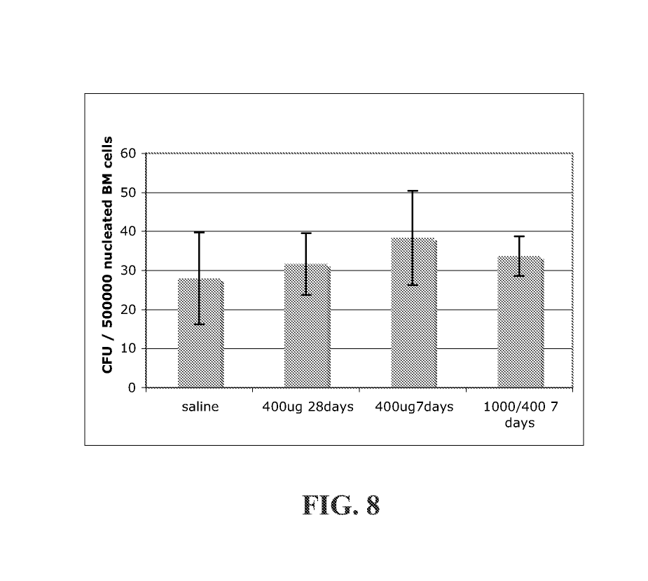

The dose resulting in 400 mcg/kg/day×7 days resulted in the greatest preservation of CFU capabilities when compared to other groups. (FIG. 8) These data are consistent with the survival data.

Infection: Of control survivors, 3/6 developed preputial inflammation with associated abscess; ⅙ in the 1000 mcg/kg dose also developed preputial inflammation and abscess. None were observed in any other groups. Cultures confirmed the presence of bacterial infection.

These data indicate a 7 day course of PTH 400 mcg/kg/day is sufficient to promote 100% 30-day survival following an LD50/30 radiation exposure. Further, such a dose preserves CFU and prevents infection when compared to non-treated groups.

### Example 7

### Effects of PTH Over Time on Irradiated Mice

B6 mice underwent 772 cGy irradiation and a daily total dose of 400 mcg/kg/day of PTH.

One group of animals was euthanized on day 3 (n=3) one group on day 7 (n=4) and one group (n=3) on day 28.

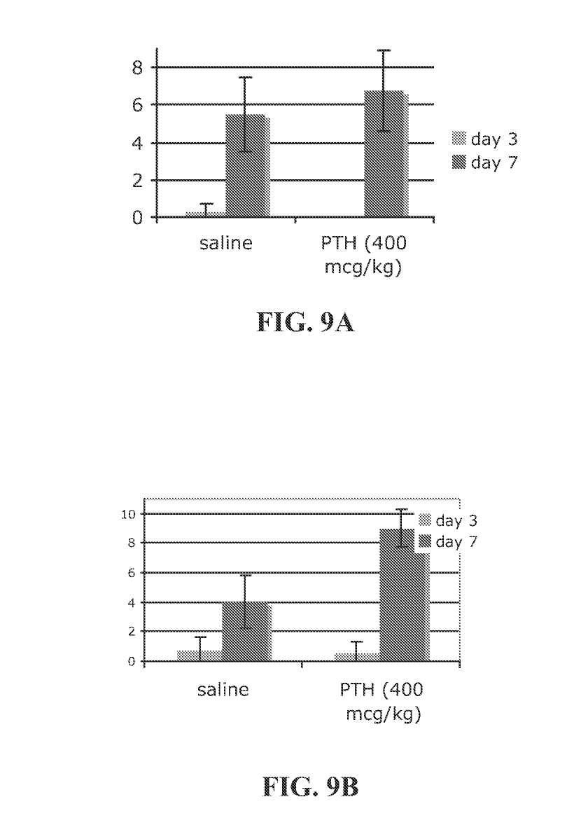

Bone marrow was analyzed for colony forming units (CFU) (FIGS. 9A and 9B), hematopoietic stem cell content. Serum was analyzed for PTH levels on the final day of the study to determine the duration of detectable PTH in the serum following the final dose.

Mice underwent exposure to 772 cGy radiation and were randomized to treatment of daily PTH 400 mcg/kg or saline. On days 3 (n=3) and 7 (n=4) bone marrow was analyzed for colony forming units. (CFU-GM and CFU-M) which increased with PTH therapy. These data show PTH has a distinctive effect on the increase of both granulocyte (GM) and monocyte (M) progenitors.

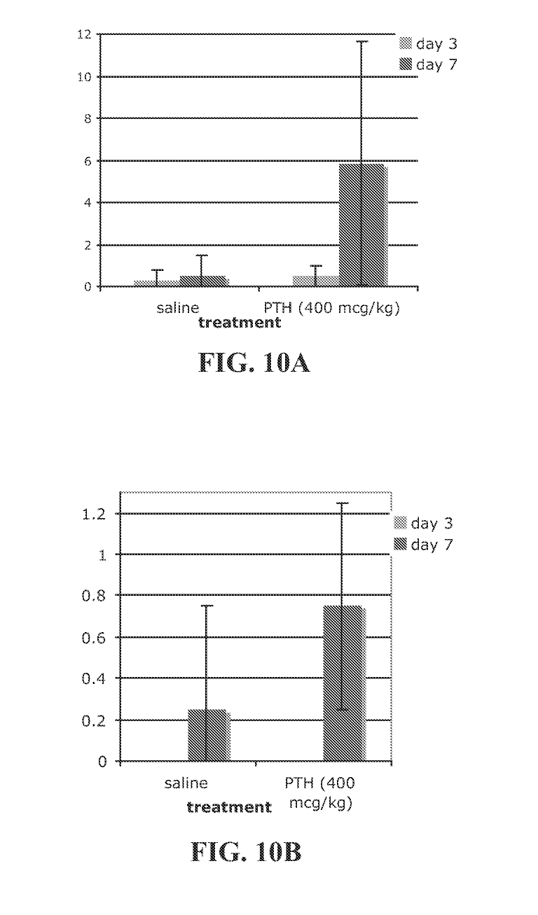

Mouse BFU (erythrocyte precursors) and CFU-GEMM indicating erythroid and megakaryoctye lineages increased with PTH treatment with a trend more pronounced on day 7 (FIGS. 10A and 10B). These data show PTH has a distinctive effect on erythroid and megakaryoctye progenitors.

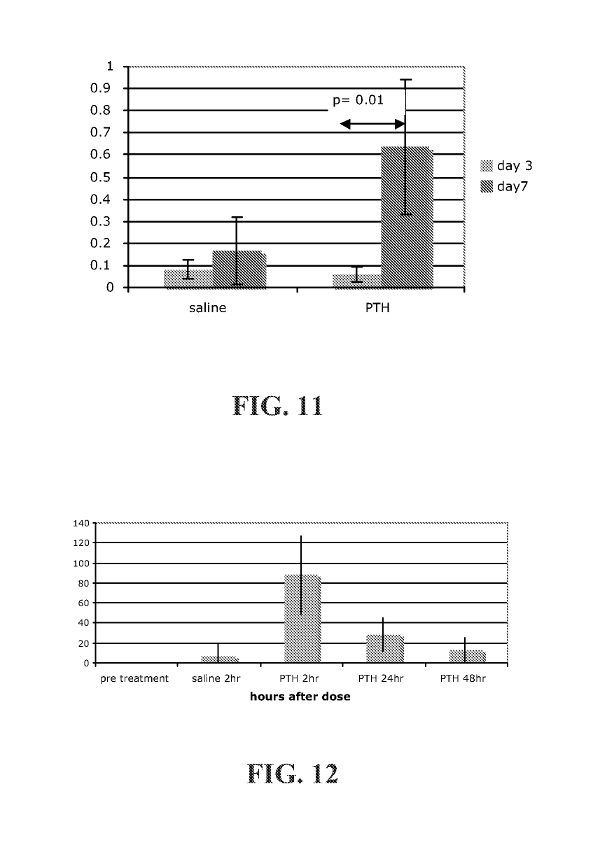

Following irradiation with 772 cGy, mice underwent bone marrow analysis on days 3 (n=3) or 7 (n=4) for hematopoietic stem cell content. While there was no statistical significance between treatment and control groups early in the course (day 3), by day 7 the PTH treated group had a significant 5-fold increase in stem cell content. (FIG. 11)

PTH detected by Mesoscale technology in the sera of mice following final PTH dose on day 28 showed some persistence of PTH at 24 hours with return to baseline at 48 hours. (FIG. 12)

### Example 8

### Effects of 2 Doses of PTH on Unirradiated Monkeys

Two cynomolgus monkeys (hereafter referred to as CYNOs) were treated with a 21 day course of daily subcutaneous PTH at either 100 mcg/kg (monkey 7801) or 50 mcg/kg (monkey 7872). Hematopoietic outcome measures included:

(a) bone marrow analysis for colony forming units 24 hours after first dose of PTH;

(b) bone marrow analysis for CD34+ hematopoietic stem cells 24 hours after first dose of PTH;

(c) serum PTH levels; and

(d) MRI of the kidneys upon completion of the 21 day course.

Cardiac monitoring showed some shortening of the Qt segment but no life threatening arrythmias.

For serum calcium, the peak did not exceed 11.5 mg/dl and returned to normal range within 6-10 hours after dosing.

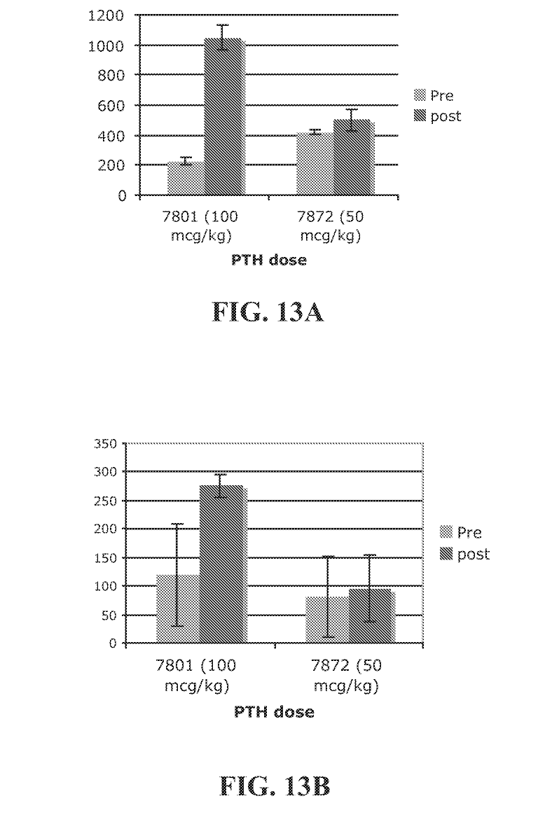

Bone marrow aspirates taken 24 hours after the first dose of PTH show increases of CFU-GM and CFU-M which appear to be more pronounced with the higher PTH dose. (FIGS. 13A and 13B)

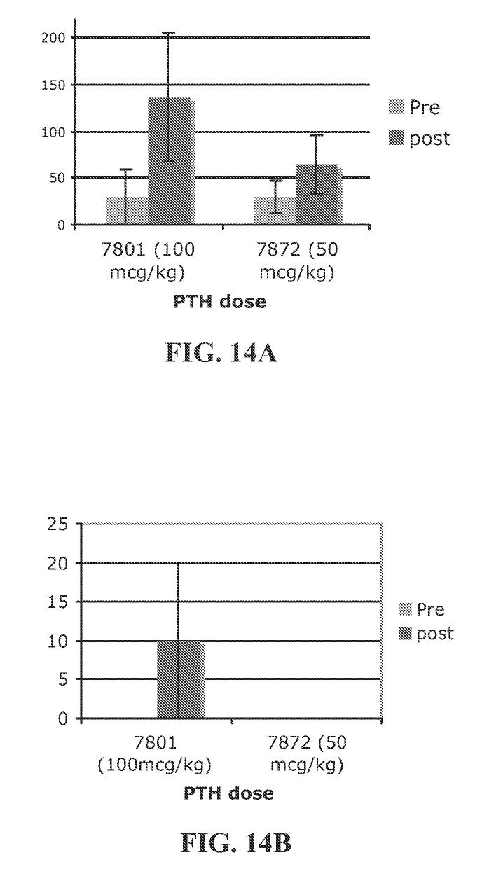

Erythroid, megakaryocyte lineages in the bone marrow aspirate also show increase after first dose of PTH. (FIGS. 14A and 14B)

Bone marrow analysis showed a dose response with higher PTH dose showing a trend for greater increases in all lineages.

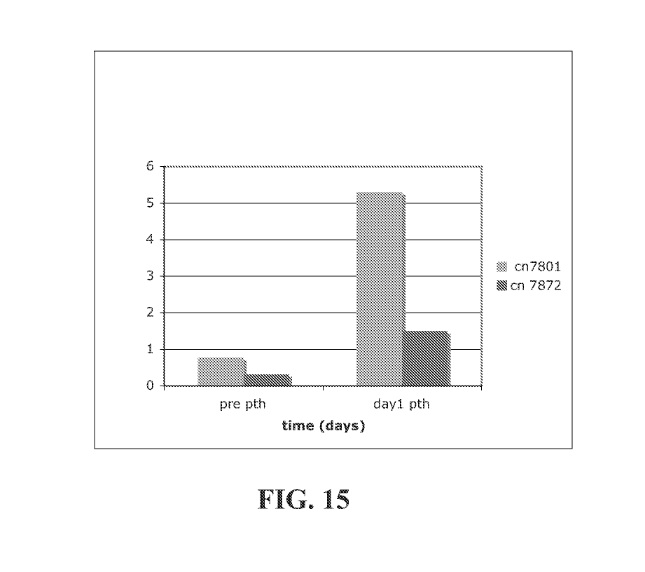

Bone aspirates analyzed 24 hours after first dose of PTH showed increases in the percent of CD34+ hematopoietic stem cells when compared to pre-treatment levels. (FIG. 15)

### Example 9

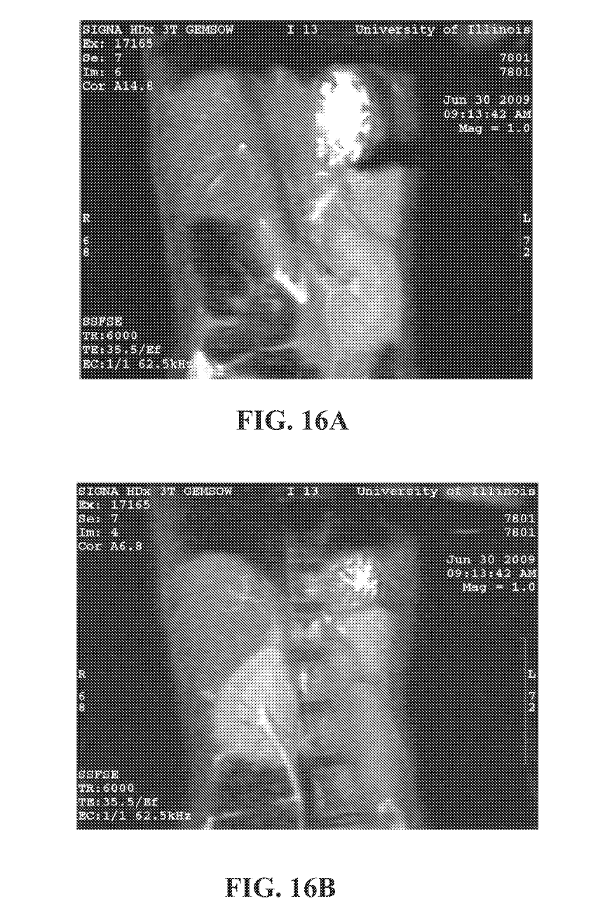

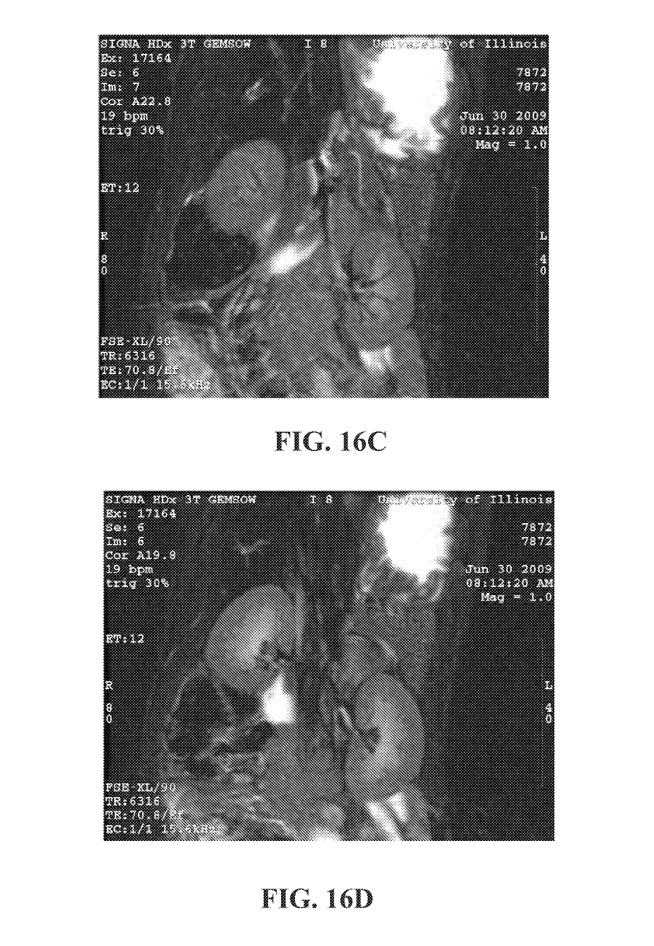

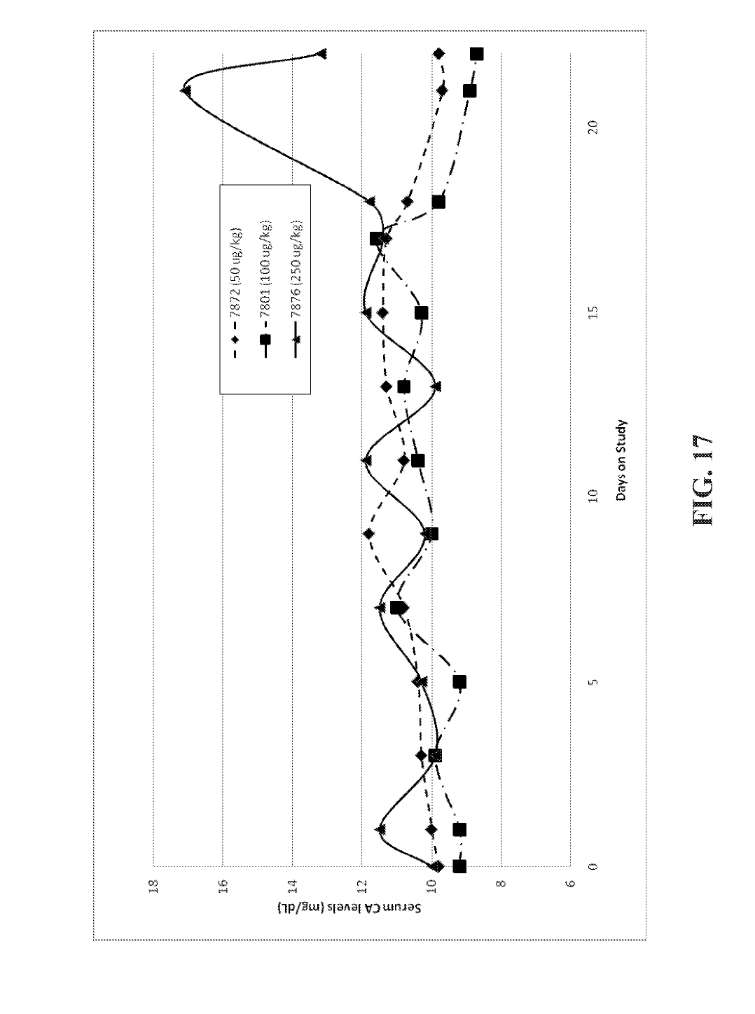

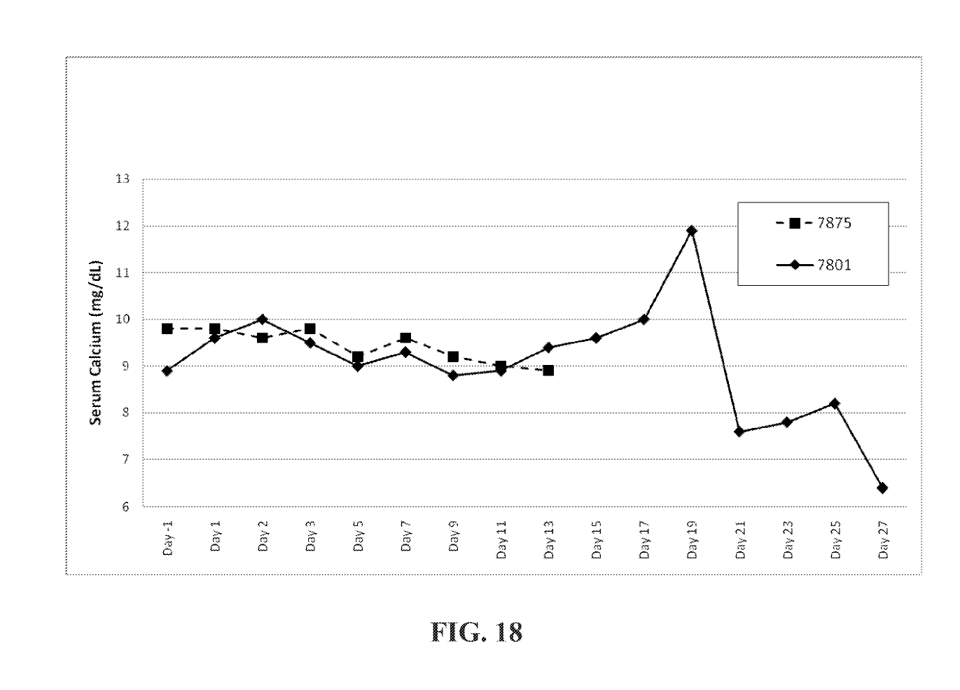

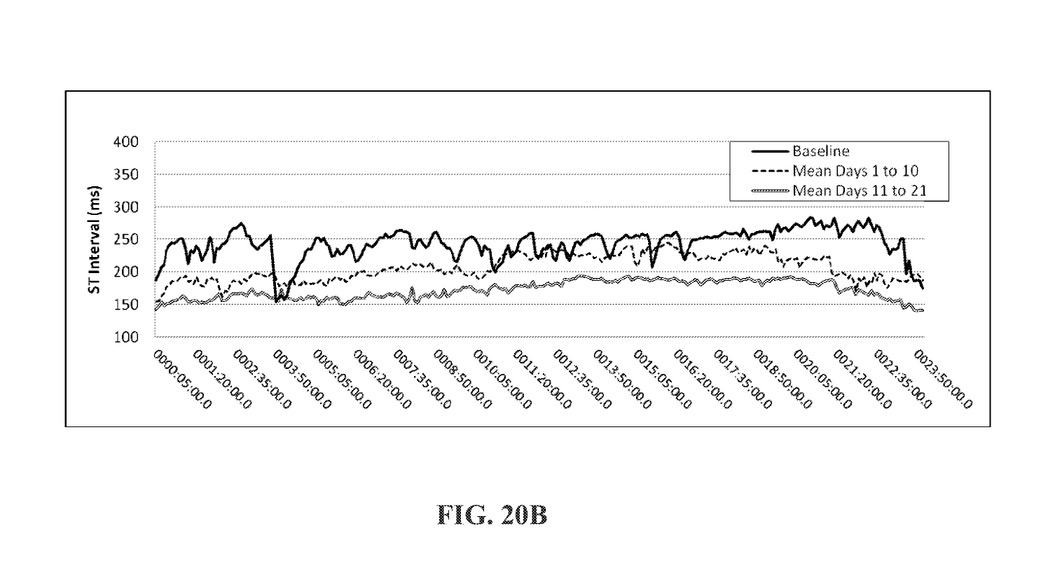

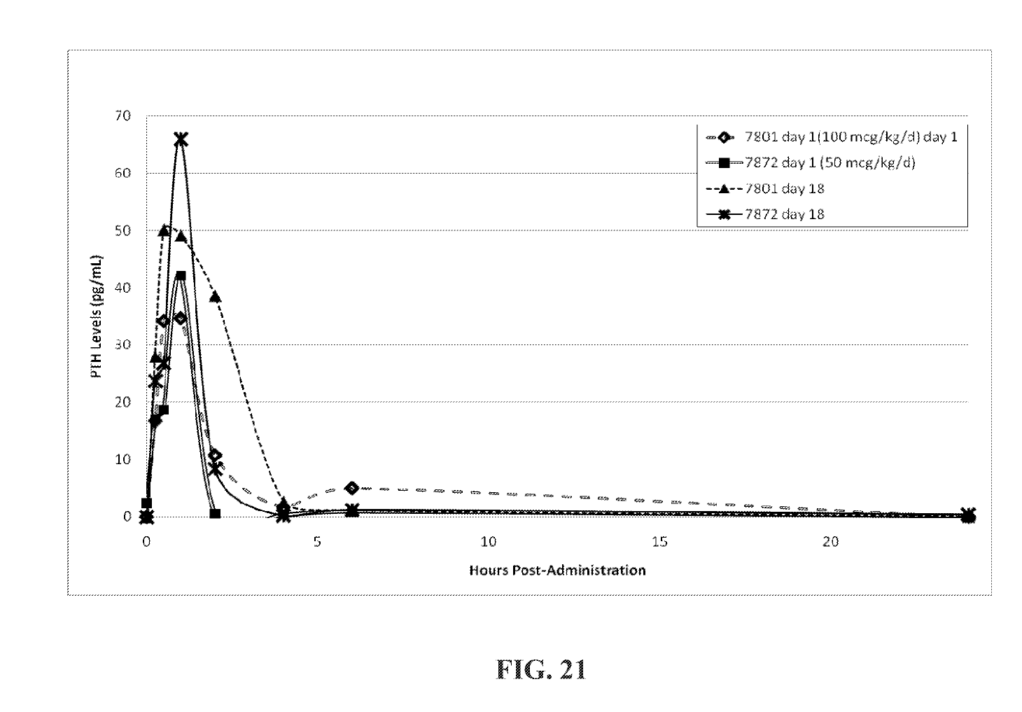

### Toxicity Studies, PTH (FIGS. 16-21)

Three cynomolgus monkeys (hereafter referred to as CYNOs) were treated with a 21 day course of daily subcutaneous PTH at either 50 ug/kg (7872), 100 ug/kg (7801), or 250 ug/kg (7876).

Two were irradiated (7801 and 7875) at a lethal dose of 682 cGy and administered 100 ug/kg. Of note, 7801 underwent a washout period prior to undergoing irradiation and subsequent PTH therapy

Outcome measures included:

(a) continuous cardiac monitoring and serum calcium

(b) serum PTH levels;

(c) bone marrow analysis for colony forming units 24 hours after first dose PTH;

(d) bone marrow analysis for CD34+ Hematopoietic stem cells 24 hours after first dose PTH; and

(e) MRI of the kidneys upon completion of the 21 day course.

### Materials and Methods

Description of Therapeutic Product

Teriparatide (Forteo), a recombinant N-terminal fragment of human PTH (rhPTH), has been approved by the US Food and Drug Administration for treatment of those with osteoporosis or at high risk of fracture. Clinical studies have found this peptide to retain comparable biological potency to the full-length PTH protein yet exert no toxic effect, even with chronic treatment.

Pharmaceutical Profile

The teriparatide raw material used in Nastech formulations is currently produced by solid phase (FMOC) peptide synthesis. The similarity of recombinant vs. synthetic teriparatide was demonstrated using peptide sequencing, LC-MS, circular dichroism, chiral analysis, specific optical rotation, UV spectroscopy, SDS-PAGE, size exclusion chromatography, isoelectric focusing, NMR, and bioassay. All data support a high degree of similarity between the recombinant and synthetic forms, and demonstrate that the synthetic PTH1-34 is similar in biological activity to the recombinant form of PTH1-34 used in Forteo. Within the stability programs of teriparatide nasal spray, no significant single impurity was observed, and the multiple lots of product have remained within all specifications up to 24 month storage at 5C. In rats and monkeys undergoing prolonged daily PTH dosing, toxicity was observed in doses exceeding 75 mcg/kg in the rat and 40 mcg/kg in monkeys. Such doses required 4 months-2 years of daily administration to achieve toxic effects. In contrast, the studies disclosed herein demonstrate higher doses of short term duration which have not been evaluated due to toxic effects observed in long-term administration and provide a new strategy for the use of PTH.

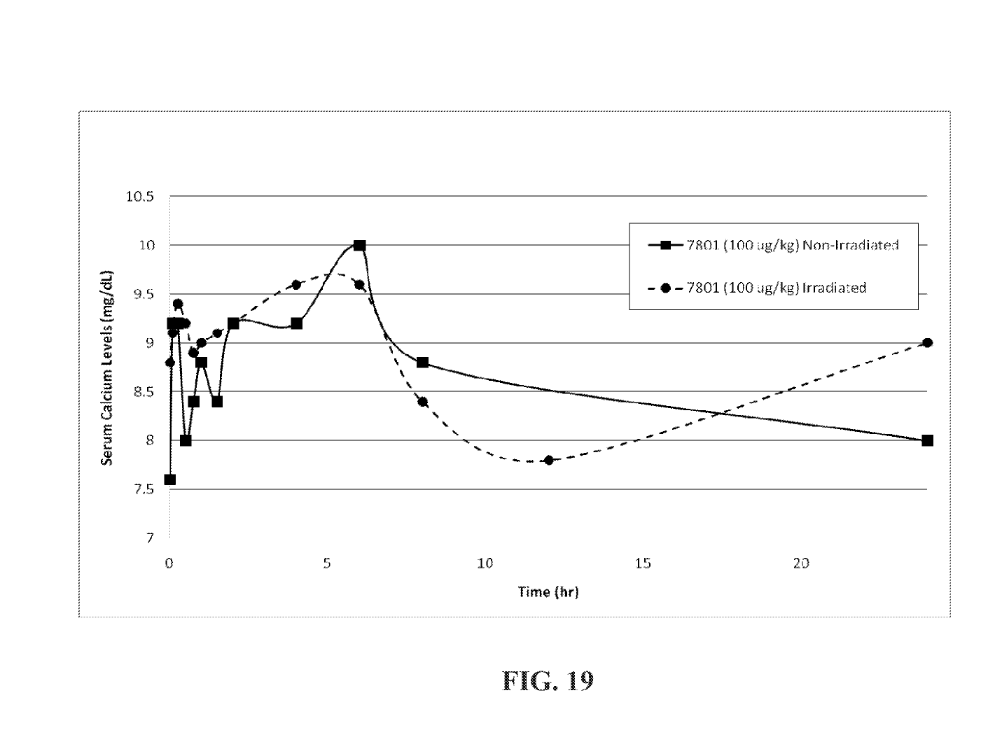

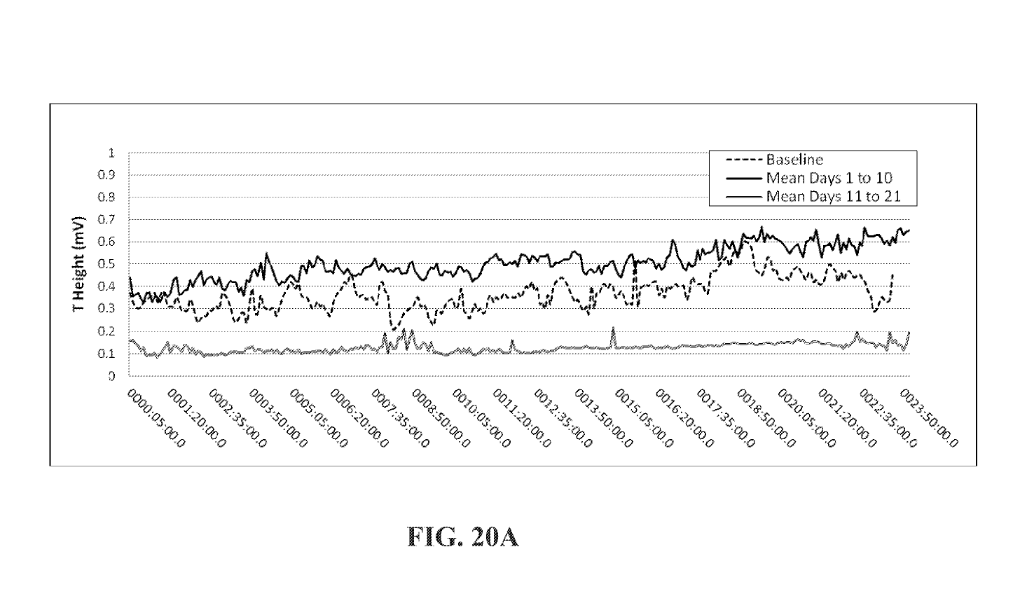

TABLE 1
Area under the Curve (AUC), Maximal Concentration (Cmax) and half-life (T½) of PTH
calculated from data presented in FIG. 19.

| | AUC0->inf | | Cmax | | T½ | |
|---|---|---|---|---|---|---|
| Study | 7872 | 7801 | 7872 | 7801 | 7872 | 7801 |
| Day | (50 μg/kg) | (100 μg/kg) | (50 μg/kg) | (100 μg/kg) | (50 μg/kg) | (100 μg/kg) |
| 1 | 47.65 | 74.58 | 42.13 | 34.64 | 14.9 | 1.7 |
| 18 | 83.5 | 119 | 65.98 | 50.1 | 5.3 | 4 |

1. A method of increasing the survival rate in a mammal exposed to acute radiation, the method comprising:
(a) obtaining a pharmaceutical composition comprising a therapeutically effective amount of parathyroid hormone (PTH) or a biologically active fragment thereof; and
(b) administering the composition to the subject to increase the survival rate of the mammal.

2. The method of claim 1, wherein the parathyroid hormone stimulates production of CD4⁺ cells and hematopoietic stem cells following acute lethal irradiation.

3. The method of claim 1, wherein the parathyroid hormone stimulates production of CD34⁺ cells.

4. The method of claim 1, wherein the fragment corresponds to an N-terminal region of human parathyroid hormone comprising about 1-34 amino acids.

5. The method of claim 1, wherein the fragment is synthetic.

6. The method of claim 1, wherein the parathyroid hormone or the fragment thereof is recombinant.

7. The method of claim 1, wherein the fragment thereof is teriparatide.

8. The method of claim 1, wherein the parathyroid hormone is administered intranasally or subcutaneously.

9. The method of claim 1, wherein the parathyroid hormone is administered intravenously or orally.

10. The method of claim 1, wherein the parathyroid hormone or the fragment thereof is administered at a dose selected from the group consisting of about 1.4 μg/kg/day to about 250 μg/kg/day.

11. The method of claim 1, wherein the radiation exposure corresponds to an exposure of 0.7-10 Gy.

12. The method of claim 1, wherein the PTH is administered for 7-50 days following an exposure to acute radiation.

13. (canceled)

14. A method to improve vascular repair and reduce end organ damage such as intestinal barrier loss or dysfunction or by alleviating acute vascular injury to a mammal exposed to radiation, the method comprising,
(a) obtaining a pharmaceutical composition comprising a therapeutically effective amount of parathyroid hormone (PTH) or a biologically active fragment thereof; and
(b) administering the composition to the subject.

15. (canceled)

16. A method to improve immune function of a mammal after exposure of said mammal to radiation, by enhanced T cell and macrophage reconstitution, including cells displaying the CD4⁺ phenotype, the method comprising:
(a) obtaining a pharmaceutical composition comprising a therapeutically effective amount of parathyroid hormone (PTH) or a biologically active fragment thereof; and
(b) administering the composition to the mammal.

17. The method of claim 16 wherein said method improves the ability of bone marrow stroma to support immune function post radiation.

18. A method of mitigating thrombocytopenia in a subject exposed to acute radiation, the method comprising:
(a) obtaining a pharmaceutical composition comprising a therapeutically effective amount of parathyroid hormone (PTH) or a biologically active fragment thereof; and
(b) administering the composition to reduce thrombocytopenia in the mammal.

19. (canceled)

20. A pharmaceutical composition comprising a therapeutically effective amount of parathyroid hormone (PTH) or a biologically active fragment thereof, wherein the PTH or the fragment thereof is capable of increasing platelets in a mammal exposed to acute radiation or other myeloablative conditions.

21. The pharmaceutical composition of claim 20, wherein the PTH or the fragment thereof comprises an aerosol formulation for a nasal spray application.

22. The pharmaceutical composition of claim 20, wherein the effective amount is in a range of about 1.4 μg/kg/day/dose to about 250 μg/kg/day/dose.

23. (canceled)

24. The method of claim 1 wherein the composition is administered intranasally, subcutaneously or orally, in a dose range of about 1.4 μg/kg/day to 250 μg/kg/day.

25. The method of claim 1 wherein the composition is administered for a time period ranging from 20-50 days following an exposure to acute radiation.

26. The method of claim 1 wherein the mammal is a human.
